(19) United States
(12) Patent Application Publication
(10) Pub. No.: US 2005/0239143 A1
(43) Pub. Date: Oct. 27, 2005

(54) Immunocapture-based measurements of mammalian pyruvate dehydrogenase complex

(76) Inventors: Roderick A. Capaldi, Eugene, OR (US); Margarita Lib, Springfield, OR (US); Michael F. Marusich, Eugene, OR (US)

Correspondence Address: KLARQUIST SPARKMAN, LLP, 121 SW SALMON STREET, SUITE 1600, PORTLAND, OR, 97204, US

(21) Appl. No.: 10/526,325

(22) PCT Filed: Aug. 29, 2003
(86) PCT No.: PCT/US03/27306

§ 371 Date: Feb. 28, 2005

(30) Foreign Application Priority Data
Aug. 30, 2002 (US) 60407376

(51) Int. Cl. G01N 33/53; G01N 33/567

(52) U.S. Cl. 435/7.2

(57) ABSTRACT

The present invention is based on the discovery that an antibody specific for PDH complex can be used to immunoprecitate PDH complex from the patient sample in an active state. Therefore the anti-PDH complex specific antibody can be used to determine the amount of and/or active state of PDH in a patient sample. The invention immunoassay methods for determining the amount and/or active state of PDH complex present in a patient sample are useful for screening to identify individuals having symptoms indicating malfunction of PDH complex. For example, the invention methods can be used to screen individuals for symptoms of onset of the diabetic state, such as insulitis, and for diagnosing late onset diseases, such as diabetes, Alzheimer's and the like.

This invention relates to immunoassays, in particular, to immunoassays for determining disorders of mitochondrial energy metabolism and diseases associated with late onset mitochondrial disorders.

### BACKGROUND OF THE INVENTION

The bulk of ATP used by many cells to maintain homeostasis is produced by the oxidation of pyruvate in the TCA cycle. During this oxidation process, reduced nicotinamide adenine dinucleotide (NADH) and reduced flavin adenine dinucleotide (FADH₂) are generated. The NADH and FADH₂ are principally used to drive the processes of oxidative phosphorylation, which are responsible for converting the reducing potential of NADH and FADH₂ to the high energy phosphate in ATP.

The fate of pyruvate depends on the cell energy charge. In cells or tissues with a high energy charge, pyruvate is directed toward gluconeogenesis, but when the energy charge is low pyruvate is preferentially oxidized to CO₂ and H₂O in the TCA cycle, with generation of 15 equivalents of ATP per pyruvate. The enzymatic activities of the TCA cycle (and of oxidative phosphorylation) are located in the mitochondrion. When transported into the mitochondrion, pyruvate encounters two principal metabolizing enzymes: pyruvate carboxylase (a gluconeogenic enzyme) and pyruvate dehydrogenase (PDH), the first enzyme of the PDH complex. With a high cell-energy charge coenzyme A (CoA) is highly acylated, principally as acetyl-CoA, and able allosterically to activate pyruvate carboxylase, directing pyruvate toward gluconeogenesis. When the energy charge is low CoA is not acylated, pyruvate carboxylase is inactive, and pyruvate is preferentially metabolized via the PDH complex and the enzymes of the TCA cycle to CO₂ and H₂O. Reduced NADH and FADH₂ generated during the oxidative reactions can then be used to drive ATP synthesis via oxidative phosphorylation.

Recently there has been renewed interest in the structure and functioning of the pyruvate dehydrogenase (PDH) complex, due to realization that altered PDH complex activity is a feature of many human disorders ranging from the relatively uncommon primary PDH deficiency [1] to major causes of morbidity and mortality, such as diabetes, starvation, sepsis and Alzheimer's disease [2-7]. PDH is a mitochondrial enzyme central to aerobic carbohydrate metabolism. It catalyzes the irreversible decarboxylation of pyruvate in the presence of CoA and NAD+ to generate CO₂, acetyl CoA and NADH [8]. PDH is one of the largest enzymes known (MW around 8,000,000) and consists of several components, each present in the complex in multiple copies. Catalytic function involves a pyruvate dehydrogenase (E1), which contains E1α and E1β subunits, dihydrolipoamide transacetylase (E2), and dihydrolipoamide dehydrogenase (E3) [8-10]. One structural subunit of the PDH complex has been described, the E3 binding protein (E3B P), which contributes to the proper assembly of the complex by promoting the interaction between E2 and E3 components [10].

More particularly, the PDH complex is comprised of multiple copies of 3 separate enzymes: pyruvate dehydrogenase (20-30 copies), dihydrolipoyl transacetylase (60 copies) and dihydrolipoyl dehydrogenase (6 copies). The complex also requires 5 different coenzymes: CoA, NAD⁺, FAD⁺, lipoic acid and thiamine pyrophosphate (TPP). Three of the coenzymes of the complex are tightly bound to enzymes of the complex (TPP, lipoic acid and FAD⁺) and two are employed as carriers of the products of PDH complex activity (CoA and NAD⁺).

The first enzyme of the complex is PDH itself, which oxidatively decarboxylates pyruvate. During the course of the reaction, the acetyl group derived from decarboxylation of pyruvate is bound to TPP. The next reaction of the complex is the transfer of the 2-carbon acetyl group from acetyl-TPP to lipoic acid, the covalently bound coenzyme of lipoyl transacetylase. The transfer of the acetyl group from acyl-lipoamide to CoA results in the formation of 2 sulfhydryl (SH) groups in lipoate, requiring reoxidation to the disulfide (S—S) form to regenerate lipoate as a competent acyl acceptor. The enzyme dihydrolipoyl dehydrogenase, with FAD⁺ as a cofactor, catalyzes that oxidation reaction. The final activity of the PDH complex is the transfer of reducing equivalents from the FADH₂ of dihydrolipoyl dehydrogenase to NAD⁺. The fate of NADH is oxidation via mitochondrial electron transport to produce 3 equivalents of ATP.

The reactions of the PDH complex serve to interconnect the metabolic pathways of glycolysis, gluconeogenesis and fatty acid synthesis to the TCA cycle. As a consequence, activity of the PDH complex is highly regulated by a variety of allosteric effectors and by covalent modification. Importance of the PDH complex to the maintenance of homeostasis is evident from the fact that, although diseases associated with deficiencies of the PDH complex have been observed, affected individuals often do not survive to maturity. Primary PDH deficiency is a severe disorder, affecting different tissues [21, 22]. Mutations in the E1α, E2, E3BP subunits and in PDP have been reported in PDH deficient patients [1, 23-25], but around 95% of all mutations are in the E1α subunit of the complex. The diagnosis of PDH deficiencies remains difficult. Recently an immunocytochemical method to aid initial diagnosis of these disorders has been discovered [26]. By this approach, mutations that alter expression of the E1α subunit, which is an x-linked gene, can be detected even when present in only 1-2% of cells, as in some female carriers, who are most often mosaic for the mutation.

Since the energy metabolism of highly aerobic tissues, such as the brain, is dependent on normal conversion of pyruvate to acetyl-CoA, aerobic tissues are most sensitive to deficiencies in components of the PDH complex. Most genetic diseases associated with PDH complex deficiency are due to mutations in PDH. The main pathologic result of such mutations is moderate to severe cerebral lactic acidosis and encephalopathies.

In addition, recent studies have shown that there is evidence of a role of mitochondrial dysfunction as a direct cause of and/or as a complication of a number of late-onset diseases. Alteration of OXPHOS functioning due to reduced synthesis and/or posttranslational modification of component proteins (and mtDNA) is now thought to be a major contributor to Parkinson's disease, Huntington's disease, Alzheimer's disease, Downs Syndrome, Schizophrenia, late-onset type II diabetes (also called NIDDM), and even the aging process itself Moreover, altered OXPHOS can also be an unintended consequence and complication of the treatment of human diseases; for example, reperfusion injury is a problem for heart attack victims and a critical issue in all organ transplants. Re-oxygenation of tissue that has become anaerobic by a cut-off of blood supply produces high concentrations of toxic-free radicals as this strong oxidant reacts with the highly reduced OXPHOS proteins, and it is this process that is thought to kill cells. Therapy for HIV infection with nucleoside reverse transcriptase inhibitors, such as AZT and DDC, causes a myopathy and lipidopathy in many patients due to a loss of oxidative OXPHOS function resulting from the reduction of mitochondrial protein synthesis. The myopathy that is an occasional side effect of statin use to treat hypercholesterolemia has also been attributed to mitochondrial toxicity of these drugs. A general discussion of research into the molecular bases of mitochondrially-related health disorders is found in Lib et al. (2002) Journal of Histochemistry and Cytochemistry 50:877-884 and Hanson et al. (2002) Journal of Histochemistry and Cytochemistry 50: 1281-288.

Therefore, there is a need in the art for new and better methods for measuring the amount and active state of PDH complex in patient samples, particularly in a format that is compatible with high throughput screening techniques.

### SUMMARY OF THE INVENTION

The present invention is predicated on the discovery that an antibody specific for PDH complex can be used to immunoprecitate PDH complex from a patient sample in an active state. Therefore the anti-PDH complex specific antibody can be used to determine the amount of and/or active state of PDH in a patient sample. The invention immunoassay methods for determining the amount and/or active state of PDH complex present in a patient sample are useful for screening to identify individuals having symptoms indicating malfunction of PDH complex. For example, the invention methods can be used to screen individuals for symptoms of onset of the diabetic state, such as insulitis, and for diagnosing late onset diseases, such as diabetes, Alzheimer's and the like.

Accordingly, in one embodiment the invention provides methods for determining the amount of pyruvate dehydrogenase (PDH) complex in a biological sample by contacting a sample comprising PDH complex with an isolated antibody that specifically binds to PDH complex under conditions to allow specific binding of the antibody to solubilized PDH complex present in the sample to form an immunocomplex. The immunocomplex is separated from remaining sample contents and the amount of the PDH complex in the separated immunocomplex is determined, thereby determining the amount of the PDH complex in the patient sample.

In another embodiment, the invention provides methods for measuring activity of PDH complex in a sample. In this procedure a sample comprising PDH complex is contacted with an isolated antibody that specifically binds to PDH complex under conditions to allow formation of an immunocomplex of the antibody and the PDH complex present in the sample and the immunocomplex is contacted with a reaction mixture comprising a non-limiting amount of substrates necessary for activity of the PDH complex. Detection of the amount of NADH produced in the reaction mixture indicates the active state of the PDH complex in the sample.

In yet another embodiment, the invention provides methods for determining the level of activity of PDH complex in a sample by measuring its level of phosphorylation. In this embodiment of the invention methods, an immunocomplex is formed as described herein and contacted with a reaction mixture comprising a non-limiting amount of substrates necessary for activity of the PDH complex. The remaining sample contents are separated from the immunocomplex; and the level of phosphorylation of immunocomplexed PDH complex in the in the sample is detected and compared with that of an unphosphorylated PDH complex standard. A level of phosphorylation greater than that in the standard indicates a lowered level of activity, and a level of phosphorylation substantially equal to that of the PDH complex in the sample indicates a normal level of activity of the PDH complex in the sample.

In still another embodiment, the invention provides methods for screening to detect an active agent that modifies inhibitor or activator activity of a known inhibitor or activator of PDH complex. A sample containing PDH complex in the presence of a known inhibitor or activator and a test active agent is contacted with a PDH complex immunoprecipitating antibody under conditions that allow formation of an antibody/PDH complex immunocomplex. The degree to which the test active agent modifies the inhibitor or activator activity of the known inhibitor or activator in the sample is detetected and compared to inhibitor or activator activity of the known inhibitor or activator in the absence of the test active agent, thereby indicating the degree to which inhibitor or activator activity is modified by the test active agent.

In a still further embodiment, the invention provides methods for screening patients to identify those suspected of having a late onset mitochondrial disorder by performing the invention immunoassay as described herein so as to detect a decrease in the amount or active state of PDH complex in a patient sample as compared with an amount or active state of PDH complex in a corresponding normal sample. A detected decrease indicates the patient is suspected of having a late onset mitochondrial disorder, such as late onset diabetes, Huntington's, Parkinson's or Alzheimer's disease, ALS (amyotrophic lateral sclerosis), or Schizophrenia.

In another embodiment, the invention provides kits containing one or more anti-PDH monoclonal antibodies that are useful for conducting the invention immunoassays.

### A BRIEF DESCRIPTION OF THE FIGURES

FIG. 1 is a graph showing activity of immunocaptured PDH as measured in moles NADH produced per 60 min. Activity was not detected when one of the substrates for the reaction was omitted. Control reaction contains the “full” reaction mixture as described in the Examples, while others lack one of the substrates or co-factors of the reaction as indicated: −CoA=minus CoA; −pyruvate=minus pyruvate; NAD=minus NAD⁺; TPP=minus thiamine pyrophosphate; control=in the presence of NAD⁺, pyruvate, CoA, TPP, MgCl₂ and cysteine as reducing agent.

FIG. 2 is a graph summarizing results of quantitation assays of saturation binding of various sized samples of human heart PDH to microtiter plates. Solubilized human heart mitochondria (HHM) was incubated with antibodies bound to 96-well microplate. After one hour of incubation, the supernatant was collected and applied to a second antibody-coated plate. After an additional one hour of incubation, the activities of PDH (residual activity) were detected and compared with those of the first plate (activity measured) for the various-sized samples.

FIG. 3 is a graph summarizing the results of tests for specific inhibition and activation of immunocaptured PDH activity. Known inhibitors and activators as shown were added directly to wells of microtiter plates and incubated with already immunocaptured PDH. Activity of the complex was monitored by measuring production of NADH per min/mg mitochondrial protein. Unmodified activity of human heart PDH (control) was set to 100%.

FIG. 4 is a graph showing the results of analysis of PDH-deficient cell lines MRC5, 404, 581 and 594 by the immunocapture microplate-based PDH activity assay. Pyruvate-dependent NADH production was followed fluorometrically in the presence of resazurin and diaphorase using an excitation wavelength of 530 nM and an emission wavelength of 590 nM. PDH activities in patient fibroblasts were calculated in relation to MRC5 control fibroblasts, where activity was set to 100%.

### DETAILED DESCRIPTION OF THE INVENTION

The present invention is predicated on the discovery that some of the mAbs used for detection of PDH subunits also immunoprecipitate the bound enzyme. Functioning of the bound enzyme was measured both spectrophotometrically or by a coupled fluorescent assay for NADH production in a high-throughput format, which is useful not only for analyzing patient samples but in screening for PDH-active drugs and environmental toxins.

The invention provides a micro scale method for the immunocapture and functional detection of active mitochondrial PDH complex from solubilized human mitochondria using a specific anti-human E-2 monoclonal antibody in an immunocapture format. The assay is suitable for high-throughput screening of samples containing mitochondria, for example obtained from human heart, human brain, human cultured fibroblast or bovine heart. The assay is specific because irrelevant antibodies fail to capture detectable activity. The assay is also quantitative and can be used to measure the amount of solubilized mitochondrial F1/F0 ATPase in samples relative to a reference control containing a known amount of PDH complex, for example. Thus, the invention assay can be used to detect disorders in production and/or utilization of PDH complex in patient samples.

The invention assays are also sensitive, requiring as little as 10 nanograms of mitochondrial protein per test, and have a wide dynamic range of at least 1000-fold. For example, when human heart mitochondria are used as a target, the assay is quantitative over a range from 10 nanograms to 10 micrograms of mitochondrial protein per sample. Thus the invention methods are suitable for use in high-throughput screening assay formats.

In one embodiment, the invention provides methods for determining the amount of pyruvate dehydrogenase (PDH) complex in a biological sample comprising providing a solid support having attached thereto antibodies specific for PDH complex; contacting the antibodies attached to the solid support with a sample comprising solubilized human fibroblasts, human heart mitochondria or bovine heart mitochondria so that the antibodies immunocapture any PDH complex present in the sample; separating remaining sample contents from the solid support; and detecting the amount of immunocaptured PDH complex on the solid support. For example, antibodies are anti-E2 specific antibodies.

In another embodiment, the invention provides methods for measuring activity of PDH complex in a sample by contacting PDH complex bound to a solid support via an antibody specific for PDH complex with a sample comprising solubilized human fibroblasts, human heart mitochondria or bovine heart mitochondria so that the antibodies immunocapture any PDH complex present in the sample. The immunocaptured PDH complex is then contacted with a reaction mixture comprising a non-limiting amount of one or more substrates necessary for activity of the PDH complex; and the amount of NADH produced in the reaction mixture is detected, wherein the amount of NADH produced indicates efficacy of biological function of the PDH complex. The detection of NADH production can be conveniently measured by transferring an electron from the reduced NADH to an electron acceptor molecule, such as an electron acceptor dye molecule to produce a change in signal from the molecule, such as absorbance of the electron acceptor dye molecule. The reaction mixture is then monitored to detect the change in signal; wherein the magnitude of the change indicates the biological activity of the PDH complex

The invention provides methods for the immunocapture and functional detection of active mitochondrial PDH complex from solubilized human or bovine mitochondria or from using a specific anti-E2 specific monoclonal antibody in an immunocapture format. The antibodies can be attached to a solid support and the assay can be conducted using high-throughput screening of samples containing mitochondria, for example obtained from human heart, human brain, human cultured fibroblast or bovine heart. The assay is specific for PDH complex, as the antibody does not capture subunits of the enzyme or 2-oxo-acid dehydrogenase

A solid support, such as a 96 well microtiter plate, coated with monoclonal anti-PDH-E2 subunit, can be used to coat the solid support with PDH for conducting activity studies. By measuring NADH production when the plate is contacted with a suitable reaction mixture containing required substrates. The assay is also quantitative and can be used to measure the amount of activity, for example on the basis of nmol NADH/min/mg of mitochondrial protein, by comparison to a reference control containing a known amount of PDH. Thus, the invention assay can be used to detect disorders in production of PDH in patient samples.

The invention assays are also sensitive, requiring as little as 50 micrograms of mitochondrial protein to saturate a 96-well microplate and have a wide dynamic range. Thus the invention methods are suitable for use in high-throughput screening assay formats.

In another embodiment, the invention PDH functional immunocapture assay is suitable for use as a diagnostic assay to detect any type of activity-affecting defect of mitochondrial PDH complex in humans, such as catalytic defects, the presence or absence of subunit antigen, defects in assembly of the enzyme complex, and the like.

In yet another embodiment of the invention, the degree of phosphorylation or dephosphorylation of PDH complex caused by an inhibitor or activator of PDH can be determined by utilizing isoelectric focusing of PDH complex, for example on a 2-D gel, to cause separation of PDH complex according to the degree of phosphorylation thereof, and visualizing the separation using a detectably labeled antibody that binds specifically to PDH complex in combination with visualization of the complex, wherein phosphorylation is indicated by an isoelectric point shift associated with a greater negative charge on the complex and dephosphorylation is indicated by an isoelectric point shift associated with a less negative charge on the PDH complex as compared with an unphorphorylated PDH complex standard.

In another embodiment, the invention provides a PDH functional immunocapture assay for determining interactions between human mitochondrial PDH complex and known inhibitors, such as sodium arsenite, or activators, such as dichloroacetate, which can be added or removed from the captured enzyme in a dose-dependent manner.

In yet another embodiment, the invention provides methods for screening to detect small molecules, drugs, or proteins that modify the inhibitor or activator activity of a known inhibitor or activator of PDH, for example by binding to the known compound so as to prevent its inhibitor or activator activity. Such small molecules, drugs, or proteins are desirable therapeutic agents that could be used to regulate activity of the PDH and thereby the energy balance and efficiency of energy utilization of cells and tissues. Such modulators have utility in treatment of disorders of energy production or utilization. Invention inhibitor screening assay comprises contacting a sample containing PDH complex in the presence of a known inhibitor and a test compound with a solid support having attached a PDH complex immunoprecipitating antibody and determining the degree to which the test compound modifies, i.e., the inhibits or activates activity of PDH complex the sample.

If combined with quantitation of captured antigen protein, the invention assays can be used to determine the specific activity (units of activity per mg enzyme protein) of a PDH complex. By this method, a distinction can be made between defects in enzyme turnover rates and defects in production of sufficient amounts of enzyme.

In still another embodiment, the invention provides isolated monoclonal antibodies characterized as specifically binding to mitochondrial PDH complex and immunoprecipitating the entire subunit complex, wherein the complex retains functional activity. Also included is a hybridoma cell line that produces a monoclonal antibody having the specificity of a monoclonal antibody as described herein.

The invention also provides a kit for determining PDH activity in a cell comprising an anti-PDH complex antibody. The kit may contain a detectable label for the antibody, such as a fluorescent label or an enzymatic label.

The assay described herein is based on the specificity of the monoclonal antibodies as well as PDH function. Any general biochemical activity of PDH function will suffice as a marker of antigen function as the antibody/antigen capture provides specificity to the assay.

In another embodiment, the invention PDH complex functional immunocapture assay is suitable for use as a diagnostic assay to detect any type of activity-affecting defect of mitochondrial PDH complex in humans, such as enzymatic defects, defects in assembly of the enzyme complex, and the like.

Therefore, the functionality of endogenous PDH complex can be determined or monitored within small samples, e.g. nanosamples. Such an assay is valuable, for example, as a research tool for studying the interactions between human mitochondrial PDH complex and its inhibitors and activators.

In yet another embodiment, the invention provides methods for screening to detect agents, such as small molecules, drugs, or proteins that modify the inhibitor or activator activity of a human mitochondrial PDH complex inhibitor or activator, for example by binding to an inhibitor so as to prevent its inhibitor activity. Such small molecules, drugs, or proteins are desirable therapeutic agents that could be used to regulate activity of PDH complex and thereby the energy balance and efficiency of energy utilization of cells and tissues. Such modulators have utility in treatment of disorders of energy production or utilization. Invention screening assays comprise contacting a sample containing PDH complex in the presence of an inhibitor or activator of PDH complex activity and a test active agent and determining the degree to which the test active agent modifies the inhibitor or activator activity of the PDH complex inhibitor or activator in the sample, wherein a decrease of inhibitor activity indicates the test active agent inhibits the activity of the PDH complex inhibitor. Active agents that increase activity of a PDH complex inhibitor or activator may also be useful in treating disorders of energy production or utilization. The invention screening assay can also be used to determine the degree to which a test active agent increases PDH complex inhibitor activity.

If combined with quantitation of captured antigen protein, the invention assays can be used to determine the specific activity (units of activity per mg enzyme protein) of PDH complex By this method, a distinction can be made between defects in enzyme turnover rates and defects in production of sufficient amounts of enzyme.

In still another embodiment, the invention provides isolated monoclonal antibodies characterized as specifically binding to mitochondrial PDH complex and immunoprecipitating the entire complex, wherein the complex retains functional activity. It should be understood that the monoclonal antibody may be able to immunoprecipitate the functional complex in the absence of all subunits being present, although the presence of all subunits is preferred.

Also included is a hybridoma cell line that produces a monoclonal antibody having the specificity of a monoclonal antibody as described herein.

The invention also provides a kit for determining PDH complex activity in a cell comprising an antibody of the invention. The kit may contain a detectable label such as a fluorescent label or an enzymatic label.

The assay described herein is based on the specificity of the monoclonal antibodies as well as antigen function. Any general biochemical activity of antigen will suffice as a marker of antigen function as the antibody/antigen capture provides specificity to the assay.

Alterations in PDH complex reduce or eliminate energy production in mitochondria and so are pathogenic. The literature shows that PDH complex is affected by a variety of environmental toxins including pesticides, impurities in narcotic drugs, and damage to organelles caused by drugs used to treat other diseases, among others. In effect these inhibit PDH complex activity, for example, enzymatic activity, to varying degrees. Mutations of PDH complex in patients (genetically derived) can also affect activity and produce the same sequellae as the toxins. Such mutations first affect and then destroy (by apoptosis) those cells with the highest need for ATP. These cells include selected brain cells such as those of the substancia nigra cells, whose impairment results in Parkinson's disease; frontal cortex cells, whose impairment is implicated in Alzheimer's disease or dementia, pancreatic cells, which are involved in insulin secretion; cardiocyte cells, whose destruction leads to cardiomyopathy, and the like. The present invention provides evidence of the utility of antibody analysis in the characterization of PDH complex deficiencies of all types.

Accordingly, in one embodiment, the invention provides methods for determining the amount of PDH complex in a biological sample of a mammalian patient. In this embodiment, the invention assay comprises contacting isolated antibodies that immunoprecipitate PDH complex with a sample comprising solubilized PDH complex so that the antibodies bind to PDH complex present in the sample to form an antibody/PDH complex immunocomplex, (i.e., under suitable conditions and for a time suitable to form the antibody/PDH complex immunocomplex). Remaining sample, for example unbound sample contents, is then separated from the immunocomplex; and the amount of PDH complex in the sample is detected.

Any suitable immunoassay format known in the art and as described herein can be used to detect and quantify the amount of antibody that binds to an antigen of interest. If the activity of the F1/F0 ATPase in the sample is also known, for example the enzymatic activity, the results of the invention method can be used to calculate the specific activity of the F1/F0 ATPase in the sample.

As used herein the term “activity” or “functional activity” as applied to F1/F0 ATPase means all aspects of natural PDH complex activity at physiological pH, including, but not limited to PDH complex enzymatic activity in oxidative phosphorylation.

In addition to antibodies that bind to fully assembled PDH complex, antibodies that are known to bind specifically to a particular PDH complex subunit can be used to determine the amount of PDH complex in a sample, if the antibody immunoprecipitates the full PDH complex. For example, an antibody that binds specifically to E-2 subunit can be used in the invention methods. Although any type of anti-PDH complex antibody, as described herein, that binds specifically to PDH complex or to an identified subunit thereof can be used in the invention methods, monoclonal antibodies are preferred.

Such assays can also be used to determine whether PDH complex is produced in low quantity as compared with what would be expected in a comparable sample obtained from a normal patient or a normal sample (i.e., obtained from a single patient that has been screened to eliminate the possibility of genetic defects in nucleotide sequences known to produce the various peptides that assemble into the PDH complex or from a representative group of such normal patients). For example, “corresponding samples” would be mitochondria isolated from a patient fibroblast cell line and mitochondria isolated from a control skin fibroblast cell line (i.e. isolated from skin fibroblasts of a normal individual). In addition to fibroblasts, PDH complex-containing samples for use in the invention methods can be obtained from whole cell extracts of the patient or from mitochondria isolated from such cells. Although fibroblast cells are particularly convenient as a source of patient samples for diagnostic assays, it should be understood that PDH complex could be isolated from any mammalian cell, including human cells, with cells having high-energy requirements having the largest supply of mitochondrial PDH complex. For example, cells that can be used in the invention methods include neural cells, cardiomyocytes, pancreatic islet cells, hematopoietic cells, liver cells, kidney cells, T cells, B cells and other cell types. Examples of tissue samples that can be utilized to obtain cells for use in the invention methods include saliva, mucosal cells and semen, for example. Alternatively, the assay can be performed utilizing PDH complex or mitochondria that have been immunopurified from patient cells or experimental cells by any method known in the art, such as the methods described in the Examples herein.

PDH complex enzymatic activity for energy production in a cell primarily depends upon the amount of functioning fully assembled PDH complex that is present in the cell. Therefore, the invention assay can be used to detect a decrease in PHD complex enzymatic activity in the cells of the patient whose sample is tested, as compared with that of a comparable normal sample.

The invention immunological tests for PDH complex activity can be used in a high throughput format using any technique known in the art, such as FASC screening as is described below in greater detail.

Detectable labels suitable for binding to antibodies used in the invention methods, including high throughput screening formats, include radiolabels linked to the antibodies using various chemical linking groups or bifunctional peptide linkers. A terminal hydroxyl can be esterified with inorganic acids, e.g., ³²P phosphate, or ¹⁴C organic acids, or else esterified to provide linking groups to the label. Enzymes of interest as detectable labels will primarily be hydrolases, particularly esterases and glycosidases, or oxidoreductases, particularly peroxidases. Fluorescent compounds include fluorescein and its derivatives, rhodamine and its derivatives, dansyl, umbelliferone, and so forth. Chemiluminescers include luciferin, and 2,3-dihydrophthalazinediones (e.g., luminol), and the like.

Antibodies may also be attached to solid supports, which are particularly useful for immunoassays or immunoprecipitation of PDH complex. Such solid supports include, but are not limited to, glass, cellulose, polyacrylamide, nylon, polystyrene, polyvinyl chloride or polypropylene, for example protein G covered wells of microtiter plates or beads.

Antibodies directed against a specific epitope, or combination of epitopes, so as to bind specifically with the PDH complex will allow for the screening of patient samples as described herein. Various screening techniques can be utilized using such monoclonal antibodies, and include magnetic separation using antibody-coated magnetic beads, “panning” with antibody attached to a solid matrix (i.e., plate), and flow cytometry (See, e.g., U.S. Pat. No. 5,985,660; and Morrison et al., Cell, 96:737-49 (1999)).

The antibodies of the invention may be assayed for immunospecific binding by any method known in the art. The immunoassays which can be used, include but are not limited to, competitive and non-competitive assay systems using techniques such as western blots, radioimmunoassays, ELISA (enzyme linked immunosorbent assay), “sandwich” immunoassays, immunoprecipitation assays, precipitin reactions, gel diffusion precipitin reactions, immunodiffusion assays, agglutination assays, complement-fixation assays, immunoradiometric assays, fluorescent immunoassays, protein A immunoassays, to name but a few. Such assays are routine and well known in the art (see, e.g., Ausubel et al, eds, 1994, Current Protocols in Molecular Biology, Vol. 1, John Wiley & Sons, Inc., New York, which is incorporated by reference herein in its entirety). Exemplary immunoassays are described briefly below (but are not intended by way of limitation).

Immunoprecipitation protocols generally comprise lysing a population of cells in a lysis buffer such as RIPA buffer (1% NP-40 or Triton X-100, 1% sodium deoxycholate, 0.1% SDS, 0.15 M NaCl, 0.01 M sodium phosphate at pH 7.2, 1% Trasylol) supplemented with protein phosphatase and/or protease inhibitors (e.g., EDTA, PMSF, aprotinin, sodium vanadate), adding the antibody of interest to the cell lysate, incubating for a period of time (e.g., 14 hours) at 4° C., adding protein A and/or protein G sepharose beads to the cell lysate, incubating for about an hour or more at 4° C., washing the beads in lysis buffer and resuspending the beads in SDS/sample buffer. The ability of the antibody of interest to immunoprecipitate a particular antigen can be assessed by, e.g., Western blot analysis. Those of skill in the art would be knowledgeable as to the parameters that can be modified to increase the binding of the antibody to an antigen and decrease the background (e.g., pre-clearing the cell lysate with sepharose beads). For further discussion regarding immunoprecipitation protocols see, e.g., Ausubel et al, eds, 1994, Current Protocols in Molecular Biology, Vol. 1, John Wiley & Sons, Inc., New York at 10.16.1.

Western blot analysis generally comprises preparing protein samples, electrophoresis of the protein samples in a polyacrylamide gel (e.g., 8%-20% SDS-PAGE depending on the molecular weight of the antigen), transferring the protein sample from the polyacrylamide gel to a membrane such as nitrocellulose, PVDF or nylon, blocking the membrane in blocking solution (e.g., PBS with 3% BSA or non-fat milk), washing the membrane in washing buffer (e.g., PBS-Tween 20), blocking the membrane with primary antibody (the antibody of interest) diluted in blocking buffer, washing the membrane in washing buffer, blocking the membrane with a secondary antibody (which recognizes the primary antibody, e.g., an anti-human antibody) conjugated to an enzymatic substrate (e.g., horseradish peroxidase or alkaline phosphatase) or radioactive molecule (e.g., ³²P or 125 I) diluted in blocking buffer, washing the membrane in wash buffer, and detecting the presence of the antigen. Those of skill in the art would be knowledgeable as to the parameters that can be modified to increase the signal detected and to reduce the background noise. For further discussion regarding western blot protocols see, e.g., Ausubel et al, eds, 1994, Current Protocols in Molecular Biology, Vol. 1, John Wiley & Sons, Inc., New York at 10.8.1.

ELISAs comprise preparing antigen, coating the well of a 96 well microtiter plate with the antigen, adding the antibody of interest conjugated to a detectable compound such as an enzymatic substrate (e.g., horseradish peroxidase or alkaline phosphatase) to the well and incubating for a period of time, and detecting the presence of the antigen. In ELISAs the antibody of interest does not have to be conjugated to a detectable compound; instead, a second antibody (which recognizes the antibody of interest) conjugated to a detectable compound may be added to the well. Further, instead of coating the well with the antigen, the antibody may be coated to the well. In this case, a second antibody conjugated to a detectable compound may be added following the addition of the antigen of interest to the coated well. Those of skill in the art would be knowledgeable as to the parameters that can be modified to increase the signal detected as well as other variations of ELISAs known in the art. For further discussion regarding ELISAs see, e.g., Ausubel et al, eds, 1994, Current Protocols in Molecular Biology, Vol. 1, John Wiley & Sons, Inc., New York at 11.2.1.

The binding affinity of an antibody to an antigen and the off-rate of an antibody-antigen interaction can be determined by competitive binding assays. One example of a competitive binding assay is a radioimmunoassay comprising the incubation of labeled antigen (e.g., ³H or ¹²⁵I) with the antibody of interest in the presence of increasing amounts of unlabeled antigen, and the detection of the antibody bound to the labeled antigen. The affinity of the antibody of interest for a particular antigen and the binding off-rates can be determined from the data by scatchard plot analysis. Competition with a second antibody can also be determined using radioimmunoassays. In this case, the antigen is incubated with antibody of interest conjugated to a labeled compound (e.g., ³H or ¹²⁵I) in the presence of increasing amounts of an unlabeled second antibody.

Antibodies used in invention assay(s) can be polyclonal, monoclonal, or a functionally active fragment thereof. Mono- or poly-clonal antibodies to a PDH isoenzyme, its salts, and PDH isoenzyme derivatives, are raised in appropriate host animals by immunization with invention immunogenic conjugate(s) using conventional techniques as are known in the art.

The preparation of monoclonal antibodies is disclosed, for example, by Kohler and Milstein, Nature 256:495-7, 1975; and Harlow et al., in: Antibodies: a Laboratory Manual, page 726 (Cold Spring Harbor Pub., 1988), which are hereby incorporated by reference. Briefly, monoclonal antibodies can be obtained by injecting mice, or other small mammals, such as rabbits, with a composition comprising an invention immunogenic conjugate whose preparation is disclosed above, verifying the presence of antibody production by removing a serum sample, removing the spleen to obtain B lymphocytes, fusing the B lymphocytes with myeloma cells to produce hybridomas, cloning the hybridomas, selecting positive clones that produce antibodies to the antigen, and isolating the antibodies from the hybridoma cultures. Monoclonal antibodies can be isolated and purified from hybridoma cultures by a variety of well-established techniques. Such isolation techniques include affinity chromatography with Protein-A Sepharose, size-exclusion chromatography, and ion-exchange chromatography. See, for example, Barnes et al., Purification of Immunoglobulin G (IgG), in: Methods in Mol. Biol., 10: 79-104, 1992). Antibodies of the present invention may also be derived from subhuman primate antibodies. General techniques for raising antibodies in baboons can be found, for example, in Goldenberg et al., International Patent Publication WO 91/11465 (1991) and Losman et al., Int. J. Cancer, 46:310-314, 1990.

It is also possible to use anti-idiotype technology to produce monoclonal antibodies which mimic an epitope. For example, an anti-idiotypic monoclonal antibody made to a first monoclonal antibody will have a binding domain in the hypervariable region which is the “image” of the epitope bound by the first monoclonal antibody.

The term “antibody” as used in this invention includes intact molecules as well as functional fragments thereof, such as Fab, F(ab′)₂, and Fv that are capable of binding a PDH isoenzyme, or a salt thereof, especially after the PDH isoenzyme or salt thereof has been derivatized with a linker molecule as disclosed herein. These functional antibody fragments are defined as follows:
(1) Fab, the fragment which contains a monovalent antigen-binding fragment of an antibody molecule, can be produced by digestion of whole antibody with the enzyme papain to yield an intact light chain and a portion of one heavy chain;
(2) Fab′, the fragment of an antibody molecule that can be obtained by treating whole antibody with pepsin, followed by reduction, to yield an intact light chain and a portion of the heavy chain; two Fab′ fragments are obtained per antibody molecule;
(3) (Fab′)₂, the fragment of the antibody that can be obtained by treating whole antibody with the enzyme pepsin without subsequent reduction; F(ab′)₂ is a dimer of two Fab′ fragments held together by two disulfide bonds;
(4) Fv, defined as a genetically engineered fragment containing the variable region of the light chain and the variable region of the heavy chain expressed as two chains; and
(5) Single chain antibody (“SCA”), a genetically engineered molecule containing the variable region of the light chain and the variable region of the heavy chain, linked by a suitable polypeptide linker as a genetically fused single chain molecule.

Methods of making these fragments are known in the art. (See for example, Harlow and Lane, Antibodies: A Laboratory Manual, Cold Spring Harbor Laboratory, New York, 1988, incorporated herein by reference). As used in this invention, the term “epitope” means any antigenic determinant on an antigen to which the paratope of an antibody binds. Epitopic determinants usually consist of chemically active surface groupings of molecules such as amino acids or carbohydrate side chains and usually have specific three dimensional structural characteristics, as well as specific charge characteristics.

Antibody fragments according to the present invention can be prepared by proteolytic hydrolysis of the antibody or by expression in E. coli of DNA encoding the fragment. Antibody fragments can be obtained by pepsin or papain digestion of whole antibodies by conventional methods. For example, antibody fragments can be produced by enzymatic cleavage of antibodies with pepsin to provide a 5S fragment denoted F(ab′)₂. This fragment can be further cleaved using a thiol reducing agent, and optionally a blocking group for the sulfhydryl groups resulting from cleavage of disulfide linkages, to produce 3.5S Fab′ monovalent fragments. Alternatively, an enzymatic cleavage using pepsin produces two monovalent Fab′ fragments and an Fe fragment directly. These methods are described, for example, by Goldenberg, U.S. Pat. Nos. 4,036,945 and 4,331,647, and references contained therein, which patents are hereby incorporated by reference in their entirety. See also Porter, R. R., Biochem. J., 73: 119-126, 1959. Other methods of cleaving antibodies, such as separation of heavy chains to form monovalent light-heavy chain fragments, further cleavage of fragments, or other enzymatic, chemical, or genetic techniques may also be used, so long as the fragments bind to the antigen that is recognized by the intact antibody.

Fv fragments comprise an association of V_{H} and V_{L} chains. This association may be noncovalent, as described in Inbar et al., Proc. Nat'l Acad. Sci. USA 69:2659-62, 1972. Alternatively, the variable chains can be linked by an intermolecular disulfide bond or cross-linked by chemicals such as glutaraldehyde. Preferably, the Fv fragments comprise V_{H} and V_{L} chains connected by a peptide linker. These single-chain antigen binding proteins (sFv) are prepared by constructing a structural gene comprising DNA sequences encoding the V_{H} and V_{L} domains connected by an oligonucleotide. The structural gene is inserted into an expression vector, which is subsequently introduced into a host cell such as E. coli. The recombinant host cells synthesize a single polypeptide chain with a linker peptide bridging the two V domains. Methods for producing sFvs are described, for example, by Whitlow and Filpula, Methods, 2: 97-105, 1991; Bird et al., Science 242:423-426, 1988; Pack et al., Bio/Technology 11:1271-77, 1993; and Ladner et al., U.S. Pat. No. 4,946,778, which is hereby incorporated by reference in its entirety.

Another form of an antibody fragment is a peptide coding for a single complementarity-determining region (CDR). CDR peptides (“minimal recognition units”) can be obtained by constructing genes encoding the CDR of an antibody of interest. Such genes are prepared, for example, by using the polymerase chain reaction to synthesize the variable region from RNA of antibody-producing cells. See, for example, Larrick and Fry, Methods, 2: 106-10, 1991.

The invention methods use monoclonal antibodies characterized as specifically binding to PDH complex of the mitochondrial respiratory chain and immunoprecipitating PDH complex, wherein PDH complex retains functional activity.

Hybridoma cell lines producing monoclonal antibodies useful in the invention methods for immunocapture of PDH complex (while allowing the PDH complex to remain enzymatically active) are commercially available by hybridoma name as used to identify the monoclonal antibodies from Molecular Probes (Eugene, Oreg.) or from the Monoclonal Antibody Facility at the University of Oregon (Eugene, Oreg.).

The invention is further illustrated by the following non-limiting examples:

### EXAMPLE 1

Materials and Methods

Monoclonal Antibodies

Monoclonal antibodies (“MAbs) used in this study were developed in the University of Oregon Monoclonal Antibody Facility, Eugene, Oreg. Anti-subunit and E2IE3 bp subunit mAbs were generated respectively by immunizing mice with purified porcine PDH complex [26]. Antibodies were screened first for binding to purified porcine PDH (Sigma) and then for specific binding to a single subunit in denaturing Western blots of both pure porcine PDH and human mitochondria.

Preparation of Heart Mitochondria

Bovine heart mitochondria were prepared as described in Hanson et al. without modifications [27]. Human heart tissue was provided and mitochondria prepared by Analytic Biological Services, Inc. according to Smith [28].

Lysis of Mitochondria

The entire procedure was performed at 4° C. in the presence of proteinase inhibitors (leupeptin 0.5 μg/ml, pepstatin 0.5 μg/ml and 1 mM PMSF). Mitochondria were diluted with PBS buffer (100 mM NaCl, 80 mM NaH₂PO₄, 20 mM NaH₂PO₄ pH 7.5) to a total concentration of 4 mg/ml in Eppendorf tubes and lysed in the presence of 0.4% lauryl maltoside. After 20 mm incubation on ice with occasional mixing, tubes were centrifuged at 14,000 rpm at 4° C. for 10 mm in an Eppendorf microfuge. The supernatant was collected and protein concentration was determined by Bradford assay. The supernatant was immediately used for immunoprecipitation and activity studies. For immunoprecipitation, the supernatant fraction was precleared by incubation of the lysed mitochondria with protein G agarose beads at 4° C. for 2 hours to minimize non-specific binding.

Cell Preparation

Normal human fibroblasts (MRC5) were obtained from the American Type Culture Collection (Manassas, Va.). Patients fibroblasts, obtained from skin biopsies of patients, were kindly provided by Dr. Nancy Kennaway, Oregon Health Sciences University, after patient consent. These included confirmed (TC404, TC581) or suspected (TC594) defects in expression of the E1α subunit. Cells were grown in high-glucose Dulbecco's modified Eagle's medium supplemented with 10% fetal calf serum and 50 μg/ml uridine. Cells were grown to confluency on 150 mm² plates, washed with calcium- and magnesium-free PBS (CMF-PBS), and than trypsinized for collection into 15 ml tubes. Harvested cells were washed with CMF-PBS 3 times and frozen at −80° C. for at least one hour. Cells than were resuspended in 200 μl of CMF-PBS with protein inhibitors (leupeptin 0.5 μg/ml, pepstatin 0.5 μg/ml and PMSF mM) and lysed in the presence of lauryl maltoside solution (total concentration 0.5%). After 30 min of incubation on ice with occasional mixing, cells were centrifuged for 20 minutes at 4° C. in Eppendorf microfuge (˜16000 g). Supernatant was saved, analyzed for protein concentration by Bradford assay and immediately used for activity measurements or for immunoprecipitation experiment.

### EXAMPLE 2

Immunoprecipitation of MRC5 Fibroblasts Bovine and Human Heart PDH Complex

All procedures were performed in the presence of protein inhibitors in the concentrations described above. The antibody column was prepared by incubating 25 mg protein G agarose beads with 80 μg/reaction of either anti-E2 specific antibody or normal mouse IgG as the negative control at room temperature for 2 hours. To couple antibodies to the protein G beads, the beads were washed twice with 0.2M sodium borate and incubated with 20 mm dimethylpimelimidate in 0.2M sodium borate for 30 mm. Then, the beads were washed twice with 0.2 ethanolamine (pH 8) and incubated in the same solution for another 2 hours to stop the reaction. Afterwards, the beads were washed 3 times with PBS buffer in the presence of proteinase inhibitors, and precleared supernatant was applied. Mitochondria were incubated with the antibody column overnight at 4° C. and the precipitated PDH complexes were washed 5 times with PBS buffer with protease inhibitors in the presence of 0.02% lauryl maltoside. Protein was released by 20 min incubation with 30-40 μl per reaction of 100 mM glycine, pH 2.5. After a brief centrifugation, the fractions were equilibrated to pH 7.5 with mM Tris-base solution, dissolved in DS-PAGE tricine sample buffer (BioRad) containing 2% β-mercaptoethanol, and the proteins separated on a 10% SDS-polyacrylamide gel at 100 V for ˜20 mm (stacking gel) and then at 200 V for ˜40 mm (separating gel) loaded on SDS-PAGE. The gel was stained by silver-staining procedure as described previously [29].

Determination of PDH Activity

96-well ELISA plates (Falcon Probind) were coated with Goat-anti-mouse IgG Fc specific antibody (0.5 μg/well) in PBS buffer overnight at 4° C. After 3 washings, wells were coated with monoclonal anti-PDH-E2 subunit at 0.5 μg/well and incubated for 1-2 hours at room temperature. The wells were washed 3-4 times and covered with a 0.5% bovine serum albumin solution for 1 hour to minimize non-specific protein binding to the wells. After blocking, wells were washed 5 times and 100 μl of solubilized mitochondria applied and incubated in the wells at room temperature for 1 hour. After incubation, the supernatant was collected and reapplied to a second antibody-covered plate to test for residual activity.

100 μl/well of the reaction mixture (50 mM Tris, pH 7.5; 2 mM β-NAD+, 225 μM TPP, 2 mM pyruvate, 150 μM Coenzyme A, 2.6 mM cysteine, 1 mM MgCl₂) was applied and NADH production was monitored either spectrophotometrically at 340 nM or by measuring fluorescence in the presence of 15 μM resazurin and 0.5 U/ml diaphorase using an excitation wavelength of 530 nm and an emission wavelength of 590 nm for up to 75 mm. Different concentrations of NADH were loaded in the presence of reaction mixture to create a standard curve on each plate. Activity was calculated as nMol NADH/min/mg of mitochondrial protein.

Results

Human Heart Bovine Heart and MRC5 Fibroblasts' Pyruvate Dehydrogenase Complexes can be Immunoprecipitated with an Anti-E2 mAb

Monoclonal antibodies raised against porcine pyruvate dehydrogenase complex were found to cross react with both human and bovine PDH on Western blots and in immunocytochemistry. One of the antibodies specific to the E2 subunit was found to immunoprecipitate the PDH complex from detergent solubilized human fibroblasts, human heart mitochondria and bovine heart mitochondria. The polypeptide profile of the immunoprecipitate was similar to that of conventionally purified mammalian PDH [30, 31] and shows 5 major bands, corresponding to subunits E2 (72 kDa), E3 (55 kDa), E3bp (50 kDa), E₁α (41 kDa) and E₁β (36 kDa), as confirmed by peptide sequencing. There was no evidence of co-precipitation of the 2-oxo-acid dehydrogenase from the gel profiles or from sequencing of bands to ensure that subunits of this enzyme were not co-migrating with PDH subunits. The immunoprecipitated complex proved to be active when bound to antibody on the beads, based on production of NADH in the presence of the required substrates and, as shown below, the immunocapture method can be adapted to measure PDH complex activity on microplates, for example, 96-well microplates.

The Activity of Immunocaptured PDH is Substrate and Co-Factor Dependent

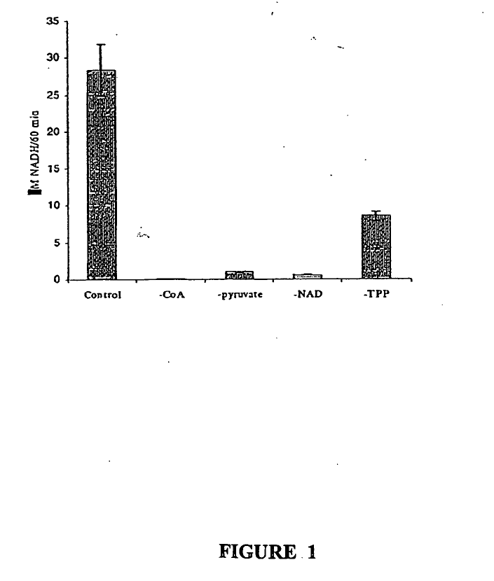

Monoclonal anti-E2 antibodies were attached to 96-well microplates to which had been prebound goat anti-mouse IgG Fc mAbs, as described in Materials and Methods. After appropriate blocking and washing, detergent-solubilized mitochondria, or cell extracts in the case of fibroblasts, were applied to the wells and incubated at room temperature for at least an hour. Unbound protein was removed by extensive washings and NADH production then measured by the increase of absorbance at 340 nM or by coupled fluorescence assay, in the presence of NAD+, pyruvate, CoA, TPP, MgCl₂, and cysteine as a reducing agent in 50 mM Tris-HCl buffer pH 7.8. FIG. 1 shows the activity of immunocaptured human heart PDH measured at 37° C. for an hour. When one of the substrates of the reaction was omitted, the absorbance remained at the background level with the exception of when TPP was omitted, where 30% total PDH activity was detected, suggesting that this co-factor is co-immunoprecipitated with the enzyme but in less than stoichiometric amounts, with respect to the E1 subunits. The same results were obtained with the fluorescent assay. As expected, the total PDH activity captured in each well of the microplate ate depends on the amount of capture mAb bound. At a fixed concentration of antibody (0.5 μg/well), there was a good linear correlation of activity with protein concentration from 6 μg to 50 μg/well (spectrophotometric measurements) or from 0.4 μg to 50 μg/well (fluorescence measurements) for both human and bovine heart mitochondria preparations.

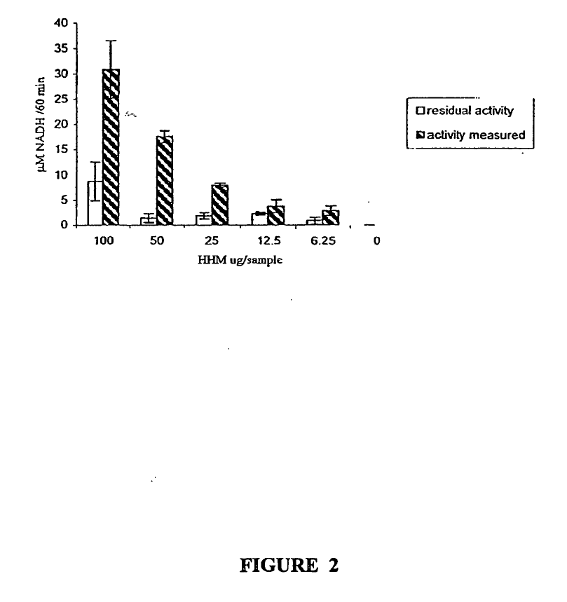

The amount of protein bound to the mAb per well was determined as follows: increasing amounts of mitochondrial protein were added to wells and allowed to bind to the capture MAb. Supernatants from each well were then transferred to a second plate coated with the same mAb. The activity captured by the second plate then measures enzyme levels in excess of what are needed to saturate the first plate (FIG. 2). At concentrations of 50 μg of mitochondrial protein, all of the activity was bound up by the first plate, but above this there was unbound enzyme for capture on the second plate. Based on this result, it can be assumed that when samples of above 50 μg are used (and excess is washed away before the assay), the activity being measured is for 50 μg protein. Thus, as shown in FIG. 2, for human heart, the PDH activity was 5.50±0.33 U, where a unit is 1 μmol of NADH produced per min/mg mitochondrial protein. The value obtained for bovine heart was 6.61±1.0 U, and these values are within the range of previously published activities for an enzyme in various purified mitochondrial preparations or tissues [32-36].

PDH can be Inhibited by Sodium Arsenite, ATP and Anti-Lipoic Acid Monoclonal Antibody

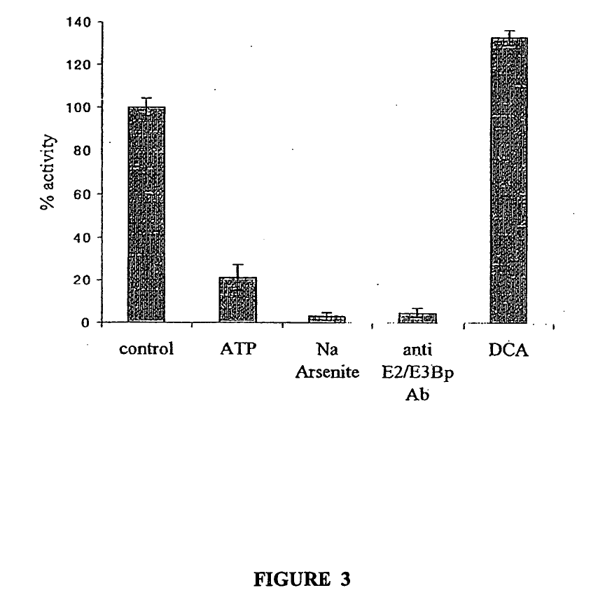

The functionality of PDH bound to the microtiter plates was further examined in the presence of several different known inhibitors and activators of the enzyme complex. Immunocaptured PDH was inhibited by sodium arsenite by >98%. In the presence of ATP, PDH activity was inhibited by about 70%. Dichloroacetate increased the activity of the enzyme by 34%. FIG. 3 summarizes the results of these studies of inhibition and activation of PDH activity. Recently, an anti-E2/E3 bp antibody has been identified as being specific for the lipoylated form of the lipoyl domain on both of these subunits. As shown, this mAb inhibited PDH activity to the background level. Thus the anti-E2/E3 antibody can be used in treatments where it is desirable to reduce PDH activity.

A Shift in Isoelectric Focusing Point can be Used to Determine the Degree of Phosphorylation of PDH

The ability of ATP to inhibit PDH suggested that the enzyme was being immunocaptured with a significant amount of PDH kinase bound. To test this further, samples were treated with ATP or dichloroactetate (DCA) and then subjected to 2D-gel electrophoresis. Previous studies have established that differently-phosphorylated forms of the E1α subunit can be resolved by the isoelectric focusing step, wherein the molecules are separated on a pH gradient by the degree of phosphorylation because addition of Pᵢ to the protein adds negative charge and shifts the spot of a phosphorylated subunit upon isoelectric focusing. [37]. Isoelectric focusing studies were conducted using a pH gradient from pH 10 to pH 3 to follow this shift for small amounts of PDH by using the E1α-specific mAb in Western blots of the 2D gels. As evident in FIG. 3, ATP reaction greatly increased the level of phosphorylation while DCA, which inhibits PDH kinases and therefore increases dephosphorylation, reduced it to below that of untreated enzyme.

Assay of PDH Activity in Patients with PDH Deficiency

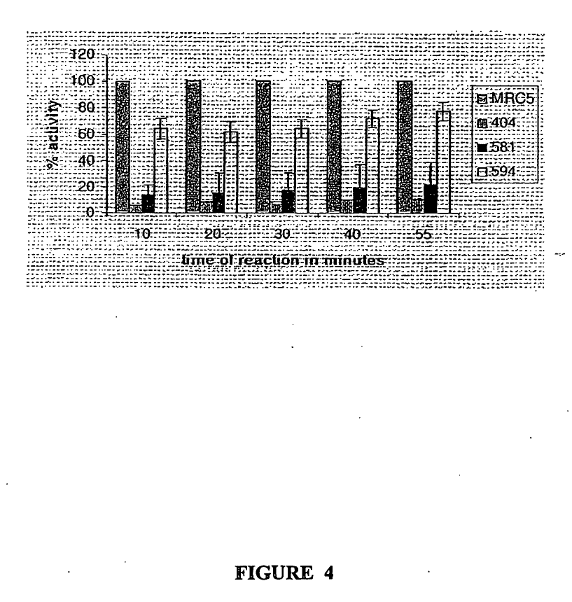

FIG. 4 shows the immunocapture assay used to detect PDH deficiency in patients. In these experiments, PDH was immunocaptured on the microtiter plates from cell lysates of human fibroblasts (MRC5) to minimize the amount of protein needed and from fibroblast cultures from 3 different patients, each with a mutation in the E1α gene. Pyruvate-dependent NADH production was followed fluorometrically using resazurin and diaphorase at an excitation wavelength of 530 um and an emission wavelength of 590 nm. The activity of the PDH in deficient fibroblasts was calculated as a percentage relative to that in normal fibroblasts (MRC5). The percent of normal PDH activity for TC404, TC594 and TC58 1 fibroblast cell lines was measured to be 7-10%, 65-78%, and 14-18% respectively, values consistent with the earlier reports of PDH activities obtained for these cell lines using the [¹⁴C] pyruvate-based activity assay [38]. The results of this study are summarized in Table 1 below:

TABLE 1
Activities of the pyruvate dehydrogenase complex in patient fibroblasts

| PDH deficient cell | PDH activity estimated in | Previously reported |
|---|---|---|
| lines | this study | PDH activity [38] |
| TC404 | 7-10% | 16-28% in lympho- |
| | | cytes |
| TC581 | 14.2-20% | 10-17% in fibroblasts |
| TC594 | 64.2%-78% | 37-41% in fibroblasts |
| | | referred to as “Just be- |
| | | low the control range” |

In combination with isoelectric focusing, the invention methods can also be used to determine the degree of phosphorylation of PDH, a mechanism that regulates glucose metabolism.

Discussion

Thus, the invention provides methods to measure PDH activity in crude mitochondrial extracts. This method is based on the ability of a monoclonal anti-E2 PDH subunit antibody to immunoprecipitate (capture) fully assembled active PDH complex from detergent-solubilized human and bovine heart mitochondrial extracts. This assay is readily adapted for use on microtiter plates, such as 96-well plates. The production of NADH in the presence of enzyme substrates can also be monitored spectrophotometrically or fluorometrically after the enzyme is captured by immobilized antibody on the well and all interfering enzyme activities were washed away. A number of methods already exist for measuring PDH activity. The most commonly used in clinical practice monitors the formation of [¹⁴C] CO₂ from radioactive pyruvate or lactate [39]. However, the use of radioactivity is difficult and limited to very few clinical centers, and is particularly problematic when high-throughput is needed. Another method described recently monitors acetyl CoA production by its reaction with a dye, acetyl CoA acrylamine-N-acetyl transferase [40]. This method requires the elaborate purification of the acrylamine-acetyltransferase from pigeon heart. Moreover, both methods measure the utilization of substrate or production formation, which for this enzyme are components metabolized by many other enzymes of the cell. Hence, many different controls are needed that are not routinely used. The invention methods provide a significant advantage in that PDH can be purified away from both other pyruvate metabolizing enzymes, as well as the many NADH-producing and NADH-utilizing enzymes in cells, which otherwise would considerably complicate PDH activity measurements.

Using the invention methods, the immunocaptured PDH complex enzyme has inhibited by previously described PDH inhibitors such as sodium arsenite and ATP [41, 42]. Also, immunoprecipitated PDH complex can be activated by DCA, a previously described PDH complex activator. [43-45]. ATP or DCA was added to immunoprecipitated bovine heart PDH and the immunocomplex was resolved on two-dimensional gel by electrophoresis over a pH range from 10 to 3. The results of this study showed that known PDH complex inhibitor ATP, not only inhibits enzyme turnover, but also increases the phosphorylation patterns of E1α. This 2-D gel electrophoresis data for anti-PDH immunoprecipitated human and bovine heart PDH complex provide evidence for the presence of both PDH kinases and PDH phosphatases in the enzyme complex as isolated using the invention methods.

Thus, the invention methods provide a new assay procedure that simplifies the diagnosis of PDH deficiencies. Heretofore, activity defects of patients have been analyzed at only a few large clinical centers set up for reproducible assay of the enzyme using [¹⁴C] pyruvate and measuring [¹⁴C] CO₂ production. The invention assays described herein are much more user friendly and provide a high throughput procedure for examining samples from multiple patients and controls at once.

The invention immunocapture method is useful in studies of the several diseases where PDH activity changes occur, e.g., diabetes [3]. Moreover, the ability to isolate the enzyme so that endogenous phosphorylation levels can be determined opens up several new avenues of research.

### REFERENCES

1. Cross, J. H., Connelly, A., Gadian, D. G., Kendall, B. E., Brown, G. K., Brown, R. M., and Leonard, J. V. (1994) Clinical diversity of pyruvate dehydrogenase deficiency. Pediair. Neurol. 10, 276-283.
2. Patel, M. S., and Harris, R. A. (1995) Mammalian alpha-keto acid dehydrogenase complexes: gene regulation and genetic defects. Faseb J. 9, 1164-1172.
3. Wu, P., Sato, I., Zhao, Y., Jaskiewicz, J., Popov, K. M., and Harris, R. A. (1998) Starvation and diabetes increase the amount of pyruvate dehydrogenase kinase isoenzyme 4 in rat heart. Biochem. J. 329 (Pt 1), 197-201.
4. Bigl, M., Bruckner, M. K., Arendt, T., Bigl, V., and Eschrich, K. (1999) Activities of key glycolytic enzymes in the brains of patients with Alzheimer's disease. J. Neural. Transm. 106, 499-511.
5. Vary, T. C., and Hazen, 5. (1999) Sepsis alters pyruvate dehydrogenase kinase activity in skeletal muscle. Mol. Cell. Biochem. 198, 113-118.
6. Heininger, K (2000) A unifying hypothesis of Alzheimer's disease. IV. Causation and sequence of events. Rev. Neurosci. 11 Spec No, 213-328.
7. Roche, T. E., Baker, J. C., Yan, X., Hiromasa, Y., Gong, X., Peng, T., Dong, I., Turkan, A., and Kasten, S. A. (2001) Distinct regulatory properties of pyruvate dehydrogenase kinase and phosphatase isoforms. Prog. Nucleic Acid Res. Mol. Biol. 70, 33-75.
8. Reed, L. J., Lawson, J. E., Niu, X. D., Yazdi, M. A., Fussey, S. P. (1992) Biochemical and molecular genetic aspects of eukaryotic pyruvate dehydrogenase multienzyme complexes. J. Nutr. Sci. Vitaminol. (Tokyo) Spec, 46-51.
9. Jilka, J. M., Rahmatullah, M., Kazemi, M., and Roche, T. E. (1986) Properties of a newly characterized protein of the bovine kidney pyruvate dehydrogenase complex. J. Biol. Chem. 261, 1858-1867.
10. Patel, M. S., and Roche, T. E. (1990) Molecular biology and biochemistry of pyruvate dehydrogenase complexes. Faseb J. 4, 3224-3233.
11. Gudi, R., Bowker-Kinley, M M., Kedishvili, N. Y., Zhao, Y., and Popov, K M. (1995) Diversity of the pyruvate dehydrogenase kinase gene family in humans. J. Biol. Chem. 270, 28989-28994.
12. Bowker-Kinley, M. M., Davis, W. I., Wu, P., Harris, R. A., and Popov, K. M. (1998) Evidence for existence of tissue-specific regulation of the mammalian pyruvate dehydrogenase complex. Biochem. J. 329 (Pt 1), 191-196.
13. Bowker-Kinley, M., and Popov, K. M. (1999) Evidence that pyruvate dehydrogenase kinase belongs to the ATPase/kinase superfamily. Biochem. J. 344 Pt 1, 47-53.
14. Wu, P., Inskeep, K., Bowker-Kinley, M. M., Popov, K. M., and Harris, R, A, (1999) Mechanism responsible for inactivation of skeletal muscle pyruvate dehydrogenase complex in starvation and diabetes. Diabetes 48, 1593-1599.
15. Baker, J. C., Yan, X., Peng, T., Kasten, S., and Roche, T. E. (2000) Marked differences between two isoforms of human pyruvate dehydrogenase kinase. J. Biol. Chem. 275, 15773-15781.
16. Steussy, C. N., Popov, K. M., Bowker-Kinley, M. M., Sloan, R. B., Jr., Harris, R. A., and Hamilton, J. A. (2001) Structure of pyruvate dehydrogenase kinase. Novel folding pattern for a serine protein kinase. J. Biol. Chem. 276, 37443-37450.
17. Sugden, P. H., Hutson, N. J., Kerbey, A. L., and Randle, P. J. (1978) Phosphorylation of additional sites on pyruvate dehydrogenase inhibits its re-activation by pyruvate dehydrogenase phosphate phosphatase. Biochem. J. 169, 433-435.
18. Sugden, P. H., and Randle, P. J. (1978) Regulation of pig heart pyruvate dehydrogenase by phosphorylation. Studies on the subunit and phosphorylation stoichiometries. Biochem. J. 173, 659-668.
19. Sugden, P. H., and Simister, N. E. (1980) Role of multisite phosphorylation in the regulation of ox kidney pyruvate dehydrogenase complex. FEBS LetL 111, 299-302.
20. Korotchkina, L. G., Khailova, L. S., and Severin, S. E. (1995) The effect of phosphorylation on pyruvate dehydrogenase. FEBS Lett. 364, 185-188.
21. Chow, C. W., and Thorburn, D. R. (2000) Morphological correlates of mitochondrial dysfunction in children. Hum. Reprod. 15 Suppl 2, 68-78.
22. Nissenkorn, A., Michelson, M., Ben-Zeev, B., and Lerman-Sagie, T. (2001) Inborn errors of metabolism: a cause of abnormal brain development. Neurology 56, 1265-1272.
23. De Vivo, D. C. (1998) Complexities of the pyruvate dehydrogenase complex. Neurology 51, 1247-1249.
24. Shany, E., Saada, A., Landau, D., Shaag, A., Hershkovitz, E., and Elpeleg, O. N. (1999) Lipoamide dehydrogenase deficiency due to a novel mutation in the interface domain. Biochem. Biophys. Res. Commun. 262, 163-166.
25. Brown, R. M., Head, R. A., and Brown, G. K. (2002) Pyruvate dehydrogenase E3 binding protein deficiency. Hum. Genet. 110, 187-191.
26. Lib, M. Y., Brown, R. M., Brown, G. K., Marusich, M. F., and Capaldi, R. A. (2002) Detection of pyruvate dehydrogenase E1 alpha-subunit deficiencies in females by immunohistochemical demonstration of mosaicism in cultured fibroblasts. J. Histochem. Cytochem. 50, 877-884.
27. Hanson, B. J., Schulenberg, B., Patton, W. F., and Capaldi, R. A. (2001) A novel subfractionation approach for mitochondrial proteins: a three-dimensional mitochondrial proteome map. Electrophoresis 22, 950-959.
28. Smith, S., Cottingham, I. R., and Ragan, C. I. (1980) Immunological assays of the NADH dehydrogenase content of bovine heart mitochondria and submitochondrial particles. FEBS Lett 110, 279-282.
29. Lauber, W. M., Carroll, J. A., Dufield, D. R., Kiesel, J. R., Radabaugh, M. R., and Malone. J. P. (2001) Mass spectrometry compatibility of two-dimensional gel protein stains. Electrophoresis 22, 906-918.
30. Roche, T. E., and Cate, R. L. (1977) Purification of porcine liver pyruvate dehydrogenase complex and characterization of its catalytic and regulatory properties. Arch. Biochem. Biophys. 183, 664-677.
31. Harris, R. A., Popov, K. M., Shimomura, Y., Zhao, Y., Jaskiewicz, J., Nanaumi, N., and Suzuki, M. (1992) Purification, characterization, regulation and molecular cloning of mitochondrial protein kinases. Adv. Enzyme Regul. 32, 267-284.
32. Hinman, L. M., and Blass, J. P. (1981) An NADH-linked spectrophotometric assay for pyruvate dehydrogenase complex in crude tissue homogenates. J. Biol. Chem. 256, 6583-6586.
33. GOHIL AND JONES, 1983
34. Sheu, K. F., Lai, J. C., Kim, Y. T., Dorante, G., and Bagg, J. (1985) Immunochemical characterization of pyruvate dehydrogenase complex in rat brain. J. Neurochem. 44, 593-599.
35. Scislowski, P. W., and Davis, E. J. (1986) A sensitive spectrophotometric assay of pyruvate dehydrogenase activity. Anal. Biochem. 155, 400-404.
36. Haas, R. H., Thompson, G., Morris, B., Conright, K., and Andrews, T. (1988) Pyruvate dehydrogenase activity in osmotically shocked rat brain mitochondria: stimulation by oxaloacetate. J. Neurochem. 50, 673-680.
37. Wicking, C. A., Scholem, R. D., Hunt, S. M., Brown, G. K. (1986) Immunochemical analysis of normal and mutant forms of human pyruvate dehydrogenase. Biochem. J. 239, 89-96.
38. Wexler, I. D., Kerr, D. S., Ho, L., Lusk, M. M., Pepin, R. A. Javed, A. A., Mole, J. E., Jesse, B. W., Thekkurnkara, T. J., Pons, G., et al. (1988) Heterogeneous expression of protein and mRNA in pyruvate dehydrogenase deficiency. Proc. Natl. Acad. Sci. USA 85, 7336-7340.
39. Schofield, P. J., Griffiths, L. R., Rogers, S. H., and Wise, G. (1980) An improved method for the assay of platelet pyruvate dehydrogenase. Clin. Chim. Acta 108, 219-227.
40. Brooks, S. P., and Storey, K. B. (1993) An improvement in the pyruvate dehydrogenase complex assay: a high-yield method for purifying arylamine acetyltransferase. Anal. Biochem. 212,452-456.
41. Walsh, D. A., Cooper, R. H., Denton, R. M., Bridges, B. J., and Randle, P. J. (1976) The elementary reactions of the pig heart pyruvate dehydrogenase complex. A study of the inhibition by phosphorylation. Biochem. J. 157, 41-67.
42. Petrick, J. S., Jagadish, B., Mash, E. A., and Aposhian, H. V. (2001) Monomethylarsonous acid (MMA(III)) and arsenite: LD(50) in hamsters and in vitro inhibition of pyruvate dehydrogenase. Chem. Res. Toxicol. 14, 651-656.
43. Evans, O. B. (1982) Dichloroacetate tissue concentrations and its relationship to hypolactatemia and pyruvate dehydrogenase activation. Biochem. Pharmacol. 31, 3124-3126.
44. Evans, O. B. (1983) Effects of dichloroacetate on brain tissue pyruvate dehydrogenase. J. Neurochem. 41, 1052-1056.
45. Gibala, M. J., and Saltin, B. (1999) PDH activation by dichloroacetate reduces TCA cycle intermediates at rest but not during exercise in humans. Am. J. Physiol. 277, 33-38.

Although the invention has been described with reference to the above examples, it will be understood that modifications and variations are encompassed within the spirit and scope of the invention. Accordingly, the invention is limited only by the following claims.

1. A method for determining the amount of pyruvate dehydrogenase (PDH) complex in a biological sample compromising:
contacting a sample comprising PDH complex with an isolated antibody that specifically binds to PDH complex under conditions to allow specific binding of the antibody to solubilized PDH complex present in the sample to form an immunocomplex;
separating remaining sample contents from the immunocomplex; and
detecting the amount of the PDH complex in the separated immunocomplex, thereby determining the amount of the PDH complex in the biological sample.

2. The method of claim 1, wherein the antibody is an anti-E2 specific antibody, a monoclonal antibody, or a monoclonal anti-E2 specific antibody.

3-5. (canceled)

6. The method of claim 1, wherein the PDH complex in the immunocomplex retains PDH activity.

7. The method of claim 1, wherein the antibody is attached to a solid support and the separating includes separating unbound sample contents from the solid support.

8. The method of claim 7, wherein the separating comprises:
(i) releasing the immunocomplex complex; and
(ii) separating the immunocomplex from other components of the sample using SDS-PAGE.

9. The method of claim 7, wherein the detecting comprises contacting immunocomplexed PDH complex with a detectable marker that binds specifically to the immunocomplexed PDH and measuring the amount of detectable marker present on the solid support.

10. The method of claim 7, wherein the solid support is a microtiter plate or beads.

11-12. (canceled)

13. The method of claim 1 further comprising:
(i) quantifying the immunocaptured PDH complex detected in the sample by comparing with a standard reference curve obtained using a purified sample of PDH complex;
(ii) determining specific activity of the immunocaptured PDH complex; or
(iii) both (i) and (ii).

14. (canceled)

15. The method of claim 13, wherein the sample is obtained from a patient sample and wherein the method further comprises distinguishing between a defect in PDH complex turnover rate and a defect in production of PDH complex in the patient.

16. A method for measuring activity of PDH complex in a sample, said method comprising:
a) contacting a sample comprising PDH complex with an isolated antibody that specifically bind to PDH complex under conditions to allow formation of an immunocomplex of the antibody and the PDH complex present in the sample;
b) contacting the immunocomplex with a reaction mixture comprising a non-limiting amount of one or more substrates necessary for activity of the PDH complex; and
c) detecting:
(i) the amount of NADH produced in the reaction mixture, wherein the amount of NADH produced indicates the active state of the PDH complex; or
(ii) the level of phosphorylation of immunocomplexed PDH complex in the in the sample as compared with that of an unphoshorylated PDH complex standard, wherein a level of phosphorylation greater than that in the standard indicates a lowered level of activity, and a level of phosphorylation substantially equal to that of the PDH complex in the sample indicates a normal level of activity of the PDH complex in the sample.

17. (canceled)

18. The method of claim 16, wherein the substrates are β-NAD⁺, Coenzyme A, FAD⁺, cysteine, pyruvate, and thiamine pyrophosphate (TPP).

19. The method of claim 16, wherein the detecting comprises:
(i) transferring an electron from reduced NADH to an electron acceptor molecule to produce NADH; and
(ii) determining a change indicating transfer of an electron to the electron acceptor molecule, wherein magnitude of the change indicates biological activity of the PDH complex.

20. The method of claim 19, wherein the electron acceptor molecule is an electron acceptor dye molecule; and wherein determining a change indicating transfer of an electron involves monitoring the reaction mixture spectrophotometrically to detect a change in absorbance of the electron acceptor dye molecule; wherein magnitude of the change indicates biological activity of the PDH complex as compared to that of a comparable healthy sample of PDH complex.

21. The method of claim 20, wherein the electron acceptor dye molecule is selected from diaphorase, resazurin, and a combination thereof.

22. The method of claim 20, wherein the monitoring comprises detecting a change in fluorescence from the dye molecule.

23. The method of claim 20, wherein the detecting comprises:
(i) contacting the reaction mixture with a PDH inhibitor and comparing an amount of resultant inhibition of the PDH complex compared to that of a comparable healthy sample of PDH complex, or
(ii) contacting the reaction mixture with a PDH complex activator and comparing an amount of resultant activation of the PDH complex compared to that of a comparable healthy sample of PDH complex.

24. The method of claim 23(i), wherein the PDH complex inhibitor is selected from sodium arsenite and ATP.

25. (canceled)

26. The method of claim 23(ii), wherein the activator is dichloroactetate.

27. A kit for use in the method of claim 16, the kit comprising an antibody specific for said PDH complex

28. (canceled)

29. The method of claim 16, further comprising:
separating remaining sample contents from the immunocomplex prior to detecting the level of phosphorylation of immunocomplexed PDH complex.

30. The method of claim 29, wherein the level of phosphorylation is compared by measuring an amount of negative isoelectric point shift of the immunocomplexed PDH complex compared to the isoelectric point of the standard, the amount of negative isoelectric point shift being directly proportional to the amount of phosphorylation of the PDH complex in the sample.

31. The method of claim 30, wherein the sample is derived from a patient and wherein the amount of negative isoelectric shift is used to screen the patient for a disorder of PDH complex activity.

32. The method of claim 31, wherein the disorder is a disorder of energy production or utilization.

33. The method of claim 32, wherein the disorder is diabetes.

34. A method for screening to detect an active agent that modifies inhibitor or activator activity of a known inhibitor or activator of PDH complex comprising:
a) contacting a sample containing PDH complex in the presence of a known inhibitor or activator and a test active agent with a PDH complex immunoprecipitating antibody under conditions that allow formation of an antibody/PDH complex immunocomplex; and
b) determining the degree to which the test active agent modifies the inhibitor or activator activity of the known inhibitor or activator in the sample as compared to inhibitor or activator activity of the known inhibitor or activator in the absence of the test active agent, thereby detecting an active agent that modifies inhibitor or activator activity of a known inhibitor or activator of PDH complex.

35. (canceled)

36. The method of claim 34, wherein:
(i) the PDH complex inhibitor is sodium arsenite or ATP and the test active agent decreases inhibitor activity of the PDH complex inhibitor; or
(ii) the PDH complex activator is dichloroactetate and the test active agent decreases activator activity of the PDH complex activator.

37. (canceled)

38. The method of claim 34, wherein the antibody is an anti-E2 specific antibody, a monoclonal antibody, or a monoclonal anti-E2 specific antibody.

39-40. (canceled)

41. A method for screening patients to identify patients suspected of having a late onset mitochondrial disorder, said method comprising:
a) contacting isolated antibodies that immunoprecipitate PDH complex with a patient sample comprising solubilized PDH complex so that the antibodies bind to solubilized PDH complex present in the sample to form an immunocomplex;
b) separating the immunocomplex from the remaining sample contents; and
c) detecting a decrease in the amount of PDH complex as compared with an amount in a corresponding normal sample, wherein the decrease indicates the patient is suspected of having the late onset mitochondrial disorder.

42. The method of claim 41, wherein the late onset mitochondrial disorder is selected from late onset diabetes, Huntington's, Parkinson's and Alzheimer's diseases, ALS (amyotrophic lateral sclerosis), and Schizophrenia.

43. The method of claim 41, wherein the separating comprises:
i) releasing the immunocomplex; and
ii) separating the immunocomplex from other components of the sample using SDS-PAGE.

44. The method of claim 41, wherein the anti-PDH complex antibodies are attached to a solid support and the antibodies are tagged with a detectable marker.

45. The method of claim 44, wherein the detecting comprises:
(i) contacting the immunocomplex with a detectable marker that binds specifically to the immunocomplex and measuring the amount of signal from the detectable marker present on the solid support, or
(ii) high throughput screening.

46. The method of claim 44, wherein the solid support is beads or a microtiter plate.

47-48. (canceled)
